(19) United States
(12) Patent Application Publication
(10) Pub. No.: US 2020/0268805 A1
(43) Pub. Date: Aug. 27, 2020

(54) COMPOSITION FOR THE TREATMENT OF A MUSCULOSKELETAL DISEASE

(71) Applicant: SCARCELL THERAPEUTICS, Paris (FR)

(72) Inventors: Antoine LAFONT, Paris (FR); Bernard COULOMB, IGNY (FR)

(21) Appl. No.: 16/345,847

(22) PCT Filed: Oct. 25, 2017
(86) PCT No.: PCT/EP2017/077336

§ 371 Date: Apr. 29, 2020

(30) Foreign Application Priority Data
Oct. 27, 2016 (FR) 1660464

(51) Int. Cl. A61K 35/38; A61P 19/02

(52) U.S. Cl. CPC A61K 35/38; A61P 19/02

(57) ABSTRACT

The present invention relates to a composition comprising buccal cells for use in the prevention or treatment of musculoskeletal diseases in horses.

### FILED OF THE INVENTION

The present invention relates to a composition useful for the prevention of treatment of a disease of the locomotor system, in particular in horses.

### BACKGROUND OF THE INVENTION

In France, the total number of horses is approximately 350,000 including approximately 28,000 racehorses. 20 to 30% of them have tendinopathies. This represents about 8000 cases per year, if we consider only horse races.

No conventional method of treatment, based on anti-inflammatories, brings real benefits compared to a natural healing in the long term, which involves a rest from 6 months to a year. In addition, these treatments are essentially symptomatic and do not cure the tendon.

It has therefore been proposed to use stem cells, in particular from bone marrow, umbilical cord or adipose tissue, in order to obtain tendon healing approaching the original tendon.

Adipose tissue cells, however, appear to differentiate less than bone marrow cells. In addition, the reliability of stem cells derived from umbilical cord is not really established.

Thus, bone marrow mesenchymal stem cells are currently used as reference treatment for horse tendinitis (Godwin et al. (2012) Equine Veterinary Journal 44:25-32).

However, bone marrow sampling is a relatively cumbersome procedure. In addition, bone marrow mesenchymal stem cells have been essentially validated for an autologous use, which means that the cells are taken from the animal to which they are administered, which adds to the cost of this method.

It would therefore be interesting to have alternative treatments, simpler and less expensive to implement.

### SUMMARY OF THE INVENTION

The present invention arises form the unexpected finding, by the inventors, that administration of a composition based on heterologous gingival fibroblasts to horses suffering from tendinitis or osteochondrosis allowed to treat these diseases.

Advantageously, gingival fibroblasts are easily sampled and prepared. In addition, they are effective even in the context of a heterologous use.

Thus, the present invention relates to a composition comprising buccal cells, in particular gingival fibroblasts, for use in the prevention or treatment of a disease of the locomotor system in an individual, in particular in a horse.

Furthermore, the present invention also relates to a method for preventing or treating a disease of the locomotor system in an individual, in particular a horse, comprising administering to the individual a composition of buccal cells, in particular of gingival fibroblasts, in a prophylactically or therapeutically effective amount.

The present invention also relates to the use of a composition comprising buccal cells, in particular gingival fibroblasts, for the preparation of a medicament for treating a disease of the locomotor system in an individual, in particular a horse.

### DETAILED DESCRIPTION OF THE INVENTION

As intended herein, the term “comprising” has the meaning of “including” or “containing”, which means that when an object “comprises” one or several elements, other elements than those mentioned may also be comprised in the object. In contrast, when an object is said to “consist of” one or several elements, the object is limited to the listed elements and cannot comprise other elements than those mentioned.

### Disease of the Locomotor System

As intended herein, a disease of the locomotor system, in particular of the horse, refers in particular to an osteo-articular or musculoskeletal disease, in particular of a limb.

Diseases of the horse locomotor system are notably described in “Maladie des Chevaux” (1994) Institut du cheval. France Agricole Editions.

Preferably, the disease of the locomotor system according to the invention is a tendinopathy or a tendon injury, in particular a tendinitis.

More preferably, the disease of the locomotor system according to the invention is a tendinitis of a flexor tendon, in particular superficial or deep of the phalanges, or of an extensor tendon, in particular lateral of the phalanges or anterior of the phalanges, especially in a horse.

As intended herein the superficial flexor tendon is also called perforated tendon and the deep tendon of the phalanges is also called perforating tendon.

Preferably also, the disease of the locomotor system according to the invention is an arthropathy.

More preferably, the disease of the locomotor system according to the invention is an osteochondrosis or an arthrosis, in particular in a horse.

As intended herein, arthrosis is synonymous with osteoarthritis.

Preferably, the osteochondrosis or arthrosis is associated with bone nodules, an osteochondritis dissecans or a bone cyst, in particular in a horse

Preferably also, the osteochondrosis or arthrosis according to the invention is at the ball, the knee, the hock at the proximal level, the hock at the distal level, or the pastern of a horse.

### Individual

Preferably, the individual according to the invention is an animal, in particular a domestic animal, more preferably selected from the group consisting of an equine, in particular a horse, of a camelid, in particular a camel, a dromedary or a llama, of a canine, in particular a dog, of a feline, in particular a cat, and of an ostrich.

The individual according to the invention can be male or female.

Preferably, the individual according to the invention is a horse, in particular a competition horse, more particularly a racehorse, a trotting horse, a driving horse, a jumping horse, a dressage horse, a cross-country horse, an eventing horse, an endurance horse or a polo horse.

The horse according to the invention may be a foal, in particular a yearling, an adult horse or an older horse.

Preferably, the individual according to the invention is a horse which has a lameness, in particular of an anterior leg or a hind leg.

### Composition

As intended herein, the expression “buccal cells” is equivalent to “cells of the oral cavity” or “cells of the oral mucosa”.

Preferably, the buccal cells according to the invention are cells of the mucosa of the cheek, of the tongue, of the palate, of the labial mucosa, of the sublingual mucosa, or of the gingiva.

Preferably, the composition according to the invention is derived from the culturing of a gum sample.

Preferably, the composition according to the invention comprises or consists of gingival fibroblasts. This is called a composition of gingival fibroblasts. The composition according to the invention may notably be a culture, possibly concentrated, of gingival fibroblasts or a suspension of gingival fibroblasts.

The procedures for taking, cultivating, and preserving gingival fibroblasts are well known to one of skill in the art and are notably described in Naveau et al. (2006) J. Periodontol. 77:238-47 and in Gogly et al. (2007) Arterioscler. Thromb. Vasc. Biol. 27:1984-90, as well as in the Examples below. In particular, the gingival fibroblasts can be obtained by culturing a sample or a biopsy of the gum, possibly after enzymatic digestion of the sample or biopsy to release the gingival fibroblasts which constitute it. Thus, the cells resulting from the culturing of a sample or biopsy of the gum are essentially gingival fibroblasts.

In one embodiment of the invention, the gingival fibroblasts according to the invention comprise mesenchymal stem cells. As one of skill in the art will well understand, the mesenchymal stem cells are then buccal mesenchymal stem cells, which means of the oral cavity or of the oral mucosa, in particular of the mucosa of the cheek, of the tongue, of the palate, of the labial mucosa, of the sublingual mucosa, or of the gingiva.

In another embodiment of the invention, the gingival fibroblasts according to the invention do not, or essentially do not, comprise mesenchymal stem cells.

Advantageously, gingival fibroblasts can be easily sampled and cultivated. In addition, gingival fibroblasts have a high growth rate.

As one of skill in the art understands well, the composition according to the invention comprises the buccal cells, in particular the gingival fibroblasts, according to the invention in a prophylactically or therapeutically effective amount. Preferably, the composition according to the invention comprises from 5 million to 40 million buccal cells, in particular gingival fibroblasts, according to the invention, more preferably from 10 million to 30 million buccal cells, in particular gingival fibroblasts, according to the invention, even more preferably from 15 to 25 million buccal cells, in particular gingival fibroblasts, according to the invention, and most preferably about 20 million buccal cells, in particular gingival fibroblasts, according to the invention. The quantification of the buccal cells, in particular the gingival fibroblasts, according to the invention may be performed by any method for counting mammalian cells known to one of skill in the art and in particular using a cell counter machine.

Preferably, the buccal cells, in particular the gingival fibroblasts, according to the invention are heterologous, which means they are taken from another individual, in particular from another horse, than the one in whom the composition is used. As one of skill in the art will well understand, the individual from whom the cells are taken and the individual to whom the cells are administered are preferably of the same species.

Preferably also, the buccal cells, in particular the gingival fibroblasts, according to the invention are autologous, which means they are taken from the same individual, in particular from the same horse, than the one in whom the composition is used.

Preferably, the composition according to the invention is administered near or at a body site to be treated, more preferably in the lesion to be treated. The administration may be performed by any method known to one of skill in the art suitable with the administration of cells. However, it is preferred that the composition is injected, in particular into the lesion to be treated. Preferably, the composition according to the invention is thus injected at a tendinitis site to be treated, more preferably in the tendinous lesion, in particular by ultrasound guidance, or in or at a joint with osteochondrosis or arthrosis, in a horse.

Preferably, the method of preventing or treating an individual according to the invention comprises the following steps:
Taking gingival fibroblasts from an individual:
Culturing the gingival fibroblasts;
Administering the gingival fibroblasts or the culture of gingival fibroblasts to the individual or to another individual.

The invention will be further explained in a non-limiting manner by the following FIGURE and Examples.

### DESCRIPTION OF THE FIGURE

FIG. 1 shows ultrasound pictures of the superficial flexor tendon of 4 horses with tendinitis of this tendon before injection of a composition of gingival fibroblasts (D0) and 3 months after the injection (D0+3 months).

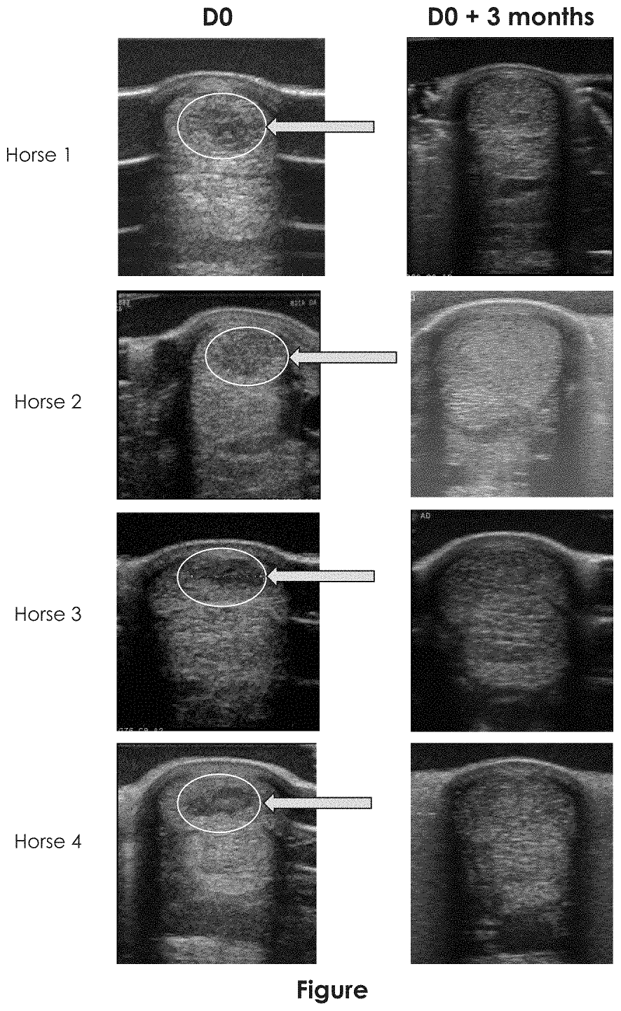

The area with the tendinous lesion, darker than its environment, is surrounded by a white circle. The injection of the composition by means of a syringe is performed in the lesion (arrow). 3 months later the tendon has a homogeneous appearance, indicating that the lesion has been treated.

### EXAMPLES

### Example 1: Treatment of a Tendinitis

### Preparation of Gingival Fibroblasts for Administration

A biopsy of approximately 50 mg of gingiva is performed with a scalpel in a horse (horse 0) and then digested enzymatically to give a suspension of gingival fibroblasts. After counting the suspension, a flask of 75 cm² is inoculated with 3×10⁵ gingival fibroblasts in complete culture medium (DMEM 4.5 g/l glucose+Glutamax (Gibco®), fetal calf serum (FCS) (Gibco®) 20%, in the presence of antibiotics/antimycotics) and is incubated at 37° C. in 5% CO₂ atmosphere up to 90% confluence by changing the culture medium regularly.

The gingival fibroblasts are then recovered. 5×10 gingival fibroblasts are used to inoculate 25 ml of complete medium in flasks of 150 cm². An incubation is then carried out at 37° C. under a 5% CO₂ atmosphere by regularly changing the culture medium until reaching 90% confluence. This forms the first passage.

2 other passages are made. At the 3rd passage, approximately 20×10⁶ gingival fibroblasts are recovered, centrifuged, washed in 10 ml of DMEM 0% FCS medium without antibiotics/antimycotics, centrifuged and taken up in a volume of approximately 3 ml. The gingival fibroblasts are then aspirated into the administration syringe and kept at 4° C. until administration.

### Administration of Gingival Fibroblasts

4 horses (horses 1, 2, 3 and 4) with tendinitis of a superficial flexor tendon, possibly associated with a lameness, have received an injection of heterologous gingival fibroblasts prepared as indicated above. The injured area of the tendon has been identified by sonography (see FIGURE). It appears as a denser (dark) area compared to its environment.

The injection of approximately 20×10⁶ gingival fibroblasts is performed directly in the lesion by ultrasound guidance (intra-lesional injection).

### Results

3 months after the injection, an ultrasound observation of the treated area is made (see FIGURE). There is no apparent tendinous lesion left: the treated area appears homogeneous by sonography. The locomotion of the animals is normal.

### Example 2: Treatment of Osteochondrosis

Gingival fibroblasts prepared as described in Example 1 are injected directly into the ball joint of a horse (horse 5) with osteochondrosis of the ball associated with a lameness.

An absence of lameness is observed approximately one week after the injection.

1. A method for the prevention or treatment of a disease of the locomotor system in a horse, comprising administering to the horse an effective amount of a composition comprising buccal cells.

2. The method according to claim 1, wherein the disease is a tendinitis in a horse.

3. The method according to claim 1, wherein the disease is tendinitis of a flexor tendon or of an extensor tendon.

4. The method according to claim 1, wherein the disease is an arthropathy.

5. The method according to claim 1, wherein the disease is an osteochondrosis or an arthrosis.

6. The method according to claim 1, wherein the horse has a lameness.

7. The method according to claim 1, wherein the composition comprises cells of the cheek, of the tongue, of the palate, of the labial mucosa, of the sublingual mucosa, or of the gingiva.

8. The method according to claim 1, wherein the composition is derived from the culturing of a gum sample.

9. The method according to claim 1, wherein the composition comprises gingival fibroblasts.

10. The method according to claim 1, wherein the composition comprises from 5 million to 40 million buccal cells.

11. The method according to claim 1, wherein the cells are heterologous, which means they are taken from another horse than the one in whom the composition is used.

12. The method according to claim 1, wherein the cells are autologous, which means they are taken from the same horse than the one in whom the composition is used.
